(19) United States
(12) Patent Application Publication
(10) Pub. No.: US 2020/0101443 A1
(43) Pub. Date: Apr. 2, 2020

(54) CATALYST FOR THE OXIDATIVE COUPLING OF METHANE WITH LOW FEED TEMPERATURES

(71) Applicant: SABIC Global Technologies, B.V., Bergen op Zoom (NL)

(72) Inventors: Wugeng LIANG, Sugar Land, TX (US); Sagar SARSANI, Sugar Land, TX (US); Hector PEREZ, Sugar Land, TX (US); David WEST, Sugar Land, TX (US); Luanyi LI, Sugar Land, TX (US)

(73) Assignee: SABIC Global Technologies, B.V., Bergen op Zoom (NL)

(21) Appl. No.: 16/582,000

(22) Filed: Sep. 25, 2019

Related U.S. Application Data
(63) Continuation of application No. 62/737,396, filed on Sep. 27, 2018.

(51) Int. Cl. B01J 23/00; B01J 23/20; B01J 23/10; B01J 35/00; C07C 2/84

(52) U.S. Cl. CPC B01J 23/002; B01J 23/20; B01J 23/10; B01J 35/0013; C07C 2/84; B01J 2523/24; C07C 2523/02; B01J 2523/3787; B01J 2523/3706; B01J 2523/3725; B01J 2523/57; C07C 2523/10; B01J 2523/3712

(57) ABSTRACT

A catalytic material for oxidative coupling of methane includes: a catalyst with the formula AₐB_{b}C_{c}Oₓ, wherein: A is selected from alkaline earth metals; B and C are selected from rare earth metals, and wherein B and C are different rare earth metals; and the oxide of at least A, B, and C has basic, redox, or both basic and redox properties, and wherein the elements A, B, and C are selected to create a synergistic effect whereby the catalytic material provides an oxygen conversion of greater than or equal to 50% and a C₂⁺ selectivity of greater than or equal to 70%, and wherein the catalyst provides the oxygen conversion and selectivity at a temperature of 797° F. (425° C.) or greater. The catalyst can be used in an oxidative coupling of methane reactor at lower feed temperatures compared to other catalysts.

### TECHNICAL FIELD

Oxidative coupling of methane (OCM) is a process whereby methane is converted into products, such as ethane and ethylene. A catalyst can be used in the presence of oxygen for the OCM reaction.

### BRIEF DESCRIPTION OF THE FIGURES

The features and advantages of certain embodiments will be more readily appreciated when considered in conjunction with the accompanying figures. The figures are not to be construed as limiting any of the preferred embodiments.

FIG. 1 is a graph of oxygen conversion (%) and C₂⁻ (%) selectivity versus reactor temperature (° C.) of a catalyst according to certain embodiments.

FIG. 2 is a graph of methane conversion (%) and ethylene selectivity versus reactor temperature (° C.) of a catalyst according to certain embodiments.

FIG. 3 is a graph of oxygen conversion (%) versus reactor temperature (° C.) to compare the oxygen conversions of different catalysts under different reactor temperatures.

FIG. 4 is a graph of C₂⁻ selectivity (%) versus reactor temperature (° C.) to compare the selectivity of the catalyst of FIG. 3.

### DETAILED DESCRIPTION OF THE INVENTION

Ethylene is crucial in the manufacture of many products, including food packaging, medical devices, lubricants, and engine coolants. Due to the crucial role ethylene plays in the production of these products, it is estimated that the production of ethylene is $160 billion per year. Heterogeneous catalysts can be used to convert methane into other products, such as ethane and ethylene, using an oxidative coupling of methane (OCM) process. In the reaction, methane (CH₄) is activated heterogeneously on the catalyst surface, forming methyl free radicals, which then couple in the gas phase to form ethane (C₂H₆). The ethane can then subsequently undergo dehydrogenation to form ethylene (C₂H₄). The oxidative coupling of methane to ethylene is shown in the equation below.

2CH₄+O₂→C₂H₄+2H₂O (Eq. 1)

However, there are many challenges in OCM to produce desirable products. For example, the yield of the desired C₂⁺ products can be reduced by non-selective reactions of the methyl radicals with the surface of the catalyst and oxygen in the gas phase, which produce undesirable products, such as carbon monoxide and carbon dioxide. Methane activation can be difficult because of its thermodynamic stability with a noble-gas-like electronic configuration. The tetrahedral arrangement of strong C—H bonds (435 kJ/mol) offer no functional group, magnetic moments, or polar distributions to undergo chemical attack. This makes methane less reactive than nearly all of its conversion products, which severely limits efficient utilization of natural gas, the world's most abundant petrochemical resource, as a source for ethylene production.

For scale-up and commercialization of OCM, some basic practical engineering problems must be solved for reactor design. It is known that there is an optimum catalyst temperature range in which C₂⁺ product selectivity is maximized, which is typically in the range from about 1,500-1,880° F. (815-1,027° C.), depending on the catalyst. At lower than optimal temperatures, oxygenated products are formed; while at higher than optimal temperatures, gasification and deep oxidation of the C₂ products start to occur. Moreover, catalyst degradation also occurs when the reactor is operated at high temperatures. Maximizing the production of desirable products requires operation at the optimal selectivity with as high methane conversion as possible. Consequently, the catalyst temperature must be controlled at some optimal and possibly narrow temperature range.

Another problem with OCM reactors is the high heat generated by the exothermic OCM reaction, which requires limiting the methane conversion to a low value in order to avoid a runaway reaction. Others have tried to solve this problem by providing a cooled multi-tubular reactor. However, such a cooled reactor that may be capable of avoiding runaway is not commercially feasible. Even with a methane to oxygen ratio of 10, such a reactor would require about 12 million tubes for a world scale ethylene plant (1,000 kTA).

An adiabatic reactor may be an alternative to a cooled reactor. An adiabatic reactor with complete oxygen conversion can have a maximum catalyst temperature as follows:

T=Tᵢₙ+ΔT_{ad}

where Tᵢₙ is the inlet temperature and T_{ad} is the adiabatic temperature rise from the exothermic reaction. The maximum cooling (by the feed) is obtained by using the lowest possible feed temperature. To avoid either catalyst deactivation or product oxidation/gasification due to too high catalyst temperature, it is necessary to limit the mole fraction of oxygen in the feed or the O₂/CH₄ ratio to some value such that the optimum catalyst temperature is not exceeded. Thus, the maximum O₂/CH₄ ratio that can be used is dictated by the inlet temperature (Tᵢₙ) and the optimum catalyst temperature. Because the per-pass methane conversion increases with increasing O₂/CH₄ ratio in the feed, methane conversion is maximized by minimizing the inlet temperature. The maximum methane conversion (minimum feed temperature) can be obtained by auto-thermal operation of an adiabatic reactor with feed at near ambient temperature. For OCM catalyst to be used for auto-thermal, adiabatic operations, special catalyst performances are needed. Thus, there is a need and an on-going industry wide concern for improved OCM catalysts that can provide comparable selectivity and conversion and be operated in reactors (e.g., adiabatic reactors) at lower feed temperatures.

It has been discovered that a catalyst can be used for the OCM to form C₂⁺ products. The catalyst possesses basic and redox properties in a ratio whereby oxygen conversion and C₂⁺ selectivity is favorable. It has also been unexpectedly discovered that the materials making up the catalyst can be used in reactors at much lower feed temperatures than expected.

As used herein, “C₂” refers to a hydrocarbon (i.e., a compound consisting of carbon and hydrogen atoms) having only two carbon atoms, for example ethane and ethylene. As used herein, the term “conversion” means the mole fraction (i.e., the percent) of a reactant converted to a product or products. As used herein, the term “selectivity” refers to the percent of converted reactant that went to a specified product (e.g., C₂⁺ selectivity is the percent of converted methane that formed C₂ and higher hydrocarbons).

It is to be understood that any discussion of the various embodiments is intended to apply to the compositions, systems, and methods.

According to certain embodiments, a catalytic material for oxidative coupling of methane comprises: a catalyst with the formula AₐB_{b}C_{c}Oₓ, wherein: A is selected from alkaline earth metals; B and C are selected from rare earth metals, and wherein B and C are different rare earth metals; and the oxide of at least A, B, and C has basic, redox, or both basic and redox properties, and wherein the elements A, B, and C are selected to create a synergistic effect whereby the catalytic material provides an oxygen conversion of greater than or equal to 50% and a C₂⁺ selectivity of greater than or equal to 70%, and wherein the catalyst provides the oxygen conversion and selectivity at a temperature of 797° F. (425° C.) or greater.

According to certain other embodiments, a system for oxidative coupling of methane comprises: a source of methane; a source of oxygen; the catalytic material, wherein the catalytic material produces ethane, ethylene, or combinations thereof; and a device for collecting or purifying the ethane, ethylene, or combinations thereof.

According to certain other embodiments, a method for the oxidative coupling of methane comprises: providing a source of methane; providing a source of oxygen; contacting the source of methane and the source of oxygen with the catalytic material, wherein the catalytic material produces ethane, ethylene, or combinations thereof after contact with the source of methane and the source of oxygen; and collecting or purifying the ethane, ethylene, or combinations thereof.

The catalyst can lower the transition state, increase the reaction rate, increase conversion of reactants, increase selectivity for a certain product, or combinations thereof, under operating conditions. According to certain embodiments, the catalyst is an OCM active catalyst and increases the rate of the OCM reaction relative to an uncatalyzed OCM reaction.

The catalyst has the general formula AₐB_{b}C_{c}Oₓ, wherein: a=1.0; b and c, and are each in the range from about 0.01 to about 10; and x is a number selected to balance the oxidation state of elements A, B and C. The catalyst can further include D_{d} having the general formula AₐB_{b}C_{c}D_{d}Oₓ, wherein d can be in the range from about 0 to about 10. It is to be understood that the general formula is meant to include the oxides of the elements A, B, C, and D and not just an oxide of D. According to certain embodiments, each of the elements A, B, C, and D is an oxide—and can be expressed as AₐOₓB_{b}OₓC_{c}OₓD_{d}Oₓ. According to certain other embodiments, some of the elements A, B, C, and D combine to form compound oxides. For example, elements A and B can form a compound oxide of AₐB_{b}Oₓ (e.g., strontium cerium oxide); or elements A, B, and C can form a compound oxide of AₐB_{b}C_{c}Oₓ (e.g., strontium cerium ytterbium oxide). The formation of compound oxides can occur during formation of the catalyst—depending on the specific elements selected. According to certain other embodiments, the element A is a nano carbonate and elements B, C, and optionally D are nano oxides. In a preferred embodiment, none of elements A, B, C, and optionally D are nitrates. This embodiment is especially beneficial because no hazardous NO and NO₂ will be produced during catalyst production. Therefore, this is an environmental friendly preparation method. Because no NO and NO₂ are formed, this preparation also make the catalyst production easier and cost less. By contrast, when nitrates are used, more is involved in the downstream preparation to address the non-environmentally friendly NOₓ formation.

Element A is selected from alkaline earth metals. According to certain embodiments, A is selected from the group consisting of magnesium, calcium, strontium, and barium; B and C are selected from the group consisting of lanthanum, scandium, cerium, praseodymium, neodymium, promethium, samarium, europium, gadolinium, yttrium, terbium, dysprosium, holmium, erbium, thulium, ytterbium, and lutetium; and D is selected from the group consisting of manganese, tungsten, bismuth, antimony, niobium, tantalum, iron, copper, or a rare earth metals, wherein if D is selected from a rare earth metal, then D is a different rare earth metal from B and C. According to certain embodiments, D is selected from the group consisting of manganese, tungsten, bismuth, antimony, and a rare earth metal (e.g., erbium, samarium, lanthanum, and neodymium).

As used herein, a “metal element” is any element, except hydrogen, selected from Groups 1 through 12 of the periodic table, lanthanides, actinides, aluminum (Al), gallium (Ga), indium (In), tin (Sn), thallium (Tl), lead (Pb), and bismuth (Bi). Metal elements include metal elements in their elemental form as well as metal elements in an oxidized or reduced state, for example, when a metal element is combined with other elements in the form of compounds comprising metal elements. For example, metal elements can be in the form of hydrates, salts, oxides, nitrates, carbonates, as well as various polymorphs thereof, and the like. As used herein, an “alkaline earth metal” is an element from Group 2 of the periodic table and includes beryllium (Be), magnesium (Mg), calcium (Ca), strontium (Sr), barium (Ba), and radium (Ra). As used herein, a “rare earth metal” is one of the fifteen lanthanides as well as scandium and yttrium, and includes cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb), and yttrium (Y). As used herein, a “transition metal” is an element from Groups 4 through 12 of the periodic table as well as scandium (Sc) and yttrium (Y) from Group 3. As used herein, a “post-transition metal” is an element located between transition metals and metalloids, and includes gallium (Ga), indium (In), thallium (Tl), tin (Sn), lead (Pb), and bismuth (Bi). A “metalloid” generally refers to the elements boron (B), silicon (Si), germanium (Ge), arsenic (As), antimony (Sb), and tellurium (Te).

The oxide of at least D has basic properties. The oxide of at least D has redox properties. The oxide of at least D can also have both basic and redox properties. The oxide of more than one, or all, of elements B, C, and D can include basic properties. The oxide of more than one of elements B, C, and D can include redox properties. One or more of elements B, C, and D can also possess both basic and redox properties.

Any of the oxides of A, B, C, and D can have different oxidation states when forming the oxide. The catalyst can be a mixture of different metal oxides and/or metal oxides having different oxidation states, to form a catalyst having two or more phases. Some of the oxides can undergo a change in oxidation state during the oxidative coupling of methane reaction. It may be that an oxide of one of the elements having basic properties, but not redox properties, can have catalytic properties without undergoing a change in oxidation state. However, the oxide of one of the elements having redox or both basic and redox properties can undergo a change in oxidation state during the reaction. For example, cerium(IV) oxide has a higher oxidation state compared to cerium(III) oxide; and thus, will be reduced by methane to form cerium(III) oxide, which in turn, becomes oxidized by oxygen to form cerium(IV) oxide. Thus, the oxidative coupling reaction can continue.

According to certain embodiments, elements A, B, C, and D are selected to create a synergistic effect whereby the catalytic material provides a desired oxygen conversion and desired C₂⁺ selectivity. The concentration (e.g., the weight concentration or molar ratios) of elements A, B, C, and D can vary. The concentration can be selected to provide a desired oxygen conversion and C₂⁺ selectivity.

According to certain embodiments, the desired oxygen conversion is greater than or equal to 50% or 90%. According to certain embodiments, the C₂⁺ selectivity is greater than or equal to 70%, 75%, or 80%. The elements A, B, C, and D and their concentrations can be selected to provide the stated oxygen conversion and C₂⁺ selectivity. It is desirable for a catalyst to provide an oxygen conversion of at least 90%. A higher oxygen conversion indicates a more active catalyst. Therefore, it is desirable to have as high an oxygen conversion as possible to have as high an activity as possible. It has been discovered that the novel catalyst can have a higher oxygen conversion at lower temperatures compared to other catalysts.

The catalytic material can be calcined. According to certain embodiments, the catalytic material is calcined at a temperature less than or equal to 2,372° F. (1,300° C.). According to certain embodiments, the catalytic material is calcined at a temperature less than or equal to 1,652° F. (900° C.). According to certain other embodiments, the catalytic material is calcined at a temperature less than or equal to 1,202° F. (650° C.).

The catalytic material can be combined with a support, binder and/or diluent material. The support and binder can be selected from any suitable material known to those skilled in the art. The diluents can be selected from bulk materials, nano materials (e.g., nanowires, nanorods, nanoparticles, etc.), and combinations thereof.

The catalytic material can be in any suitable form including, but not limited to, powder, tablet, or extruded form. Powder forms can be customized by selecting appropriate particle size distributions that provide a desired C₂⁺ selectivity and oxygen conversion. The catalyst can also have a variety of shapes including, but not limited to, cylinder, ring, flat sheet, multi-lobe cylinder, wagon wheel, etc. The catalyst can also have a variety of dimensions—depending in part, on the type of reactor used and the scale of the operation. One of ordinary skill in the art will be able to select the appropriate dimensions for the catalyst.

According to certain embodiments, the raw materials used for catalyst preparation are in the form of a nano material. As used herein, a “nano material” is a material having a mean particle size in the range from 1 to 900 nanometers (nm). This embodiment can be useful to improve oxygen conversion and C₂⁺ selectivity.

It is also desirable to provide formed catalysts having a uniform porosity. The porosity of a material is the void fraction or percent of empty spaces within a material. For example, the porosity of the formed catalyst can directly impact the efficiency of the catalyst by providing accessibility of the reactants to the catalysts surfaces where the reaction of interest is catalyzed. Variances of the porosity of the catalyst, either within a single formed catalyst particle, or between different catalyst particles, can impact the overall efficiency of the catalytic material, for example, by providing regions of low activity and regions of high activity. The regions with different activity can lead to additional issues, such as thermal non-uniformity in catalyst particles or catalyst beds. Moreover, the relative porosity of a catalyst particle can also directly impact its structural characteristics, such as crush strength, leading to catalyst particles that have relatively lower crush strength in one portion of the particle or in one particle relative to another. This difference in structural properties can again, impact catalytic processes by altering handling and processing ability, generation of fines, and other issues. According to certain embodiments, the catalytic material has a uniform porosity, which can be provided, at least in part, through the use of powdered compositions having uniform particle size distributions. The porosity of a tablet or extruded form can also be selected to provide a desired C₂⁺ selectivity and oxygen conversion. According to certain embodiments, the porosity is in the range of about 10% to about 80%.

The catalytic material, including a support, binder, or diluent, can be made by a variety of processes known to those skilled in the art. Examples of suitable processes include, but are not limited to, tableting, extrusion, and impregnation.

It has been discovered that the catalytic material according to the embodiments can be used in reactors with much lower feed temperatures compared to other catalytic materials. This lower feed temperature can overcome the issues with other catalytic materials. According to certain embodiments, the catalyst is thermally stable at a temperature of 797° F. (425° C.) or greater. The catalyst can also be thermally stable at a temperature in the range of about 797° F. (425° C.) to about 2,372° F. (1,300° C.). According to certain other embodiments, the catalyst provides the oxygen conversion and selectivity at a temperature in the range of about 797° F. (425° C.) to about 2,372° F. (1, 300° C.). One of the many advantages to the novel catalyst is the catalyst can be used in an adiabatic reactor with low feed temperature with favorable oxygen conversion and selectivity. Therefore, according to certain embodiments, the catalyst is used in an OCM adiabatic reactor with low feed temperature.

A system for oxidative coupling of methane can include a source of methane, a source of oxygen, the catalytic material, and a device for collecting the ethane, ethylene, or combinations thereof.

The system can include feed and product streams for C₂ production. A first feed stream can be the source of methane, and a second feed stream can be the source of oxygen. The source of methane can include natural gas, associated gas, and shale gas. The source of oxygen can include air, oxygen enriched air, pure oxygen, oxygen diluted with nitrogen (or another inert gas), or oxygen diluted with carbon dioxide (CO₂). A first product stream can include water and hydrogen gas, and a second product stream can include ethane (C₂H₆), ethylene (C₂H₄), and other reaction products.

Any of the feeds or products can be separated, condensed, and/or recycled back into a given feed or product stream. For example, hydrogen gas and any unreacted steam from the second product stream can be separated, collected, and stored or recycled back into the feed stream. By way of another example, the products from the product streams can be flowed through one or more distillation columns or other separators to separate C₂ products from other reaction products.

The ratio of methane to oxygen from the product stream(s) can vary. The C₂⁺ selectivity, oxygen conversion, and percent yield of C₂ products can also vary depending on the ratio of methane to oxygen for a particular catalytic material under operating conditions. According to certain embodiments, the ratio of methane to oxygen is selected to provide a percent yield of the ethane, ethylene, or combinations thereof that is greater than or equal to 10%, preferably greater than or equal to 15%. According to certain other embodiments, the ratio of methane to oxygen is in the range of about 2:1 to about 10:1. The ratio of methane to oxygen as well as the selection and concentration of A, B, C, and D can be selected to provide an increased percent yield of C₂ products. It may be desirable to produce more ethylene than ethane. Therefore, according to certain embodiments, the ratio of methane to oxygen as well as the selection and concentration of elements A, B, C, and D are selected to provide an increased percent yield of ethylene.

Methods for the oxidative coupling of methane can include providing a source of methane, providing a source of oxygen, contacting the source of methane and the source of oxygen with the catalytic material, and collecting the ethane, ethylene, or combinations thereof.

The operating temperature for the OCM reaction can be in the range of about 572° F. (300° C.) to about 2,372° F. (1,300° C.) or in the range of about 932° F. (500° C.) to about 2,012° F. (1,100° C.). The operating pressure can be in the range of about 1 bar to about 100 bars or in the range of about 1 bar to about 10 bars. The gas hourly space velocity (GHSV) for the OCM reaction can range from about 500 hr⁻¹ to 5,000,000 hr⁻¹, or from 5000 hr⁻¹ to 1,000,000 hr⁻¹.

### EXAMPLES

To facilitate a better understanding of the present invention, the following examples of certain aspects of preferred embodiments are given. The following examples are not the only examples that could be given according to the present invention and are not intended to limit the scope of the invention.

Catalysts having the general formula AₐB_{b}C_{c}Oₓ or AₐB_{b}C_{c}D_{d}Oₓ were prepared according to the following methods. The example catalysts were prepared by using nano raw materials, the catalysts were formed by placing a known amount of the nano materials in a porcelain dish. A known amount of deionized water was added to make a slurry to enhance the mixing of the nano materials. The mixtures were then dried at a temperature of about 125° C. for at least 8 hours. The cakes were then placed in a heating oven for calcination at various calcination temperatures for 6 hours. Pressing is optionally included if after calcination the catalyst material is in a powder form. The solid catalysts were crushed to powder and sieved to form a product having a particle size between 35 to 60 mesh for performance testing. For the example catalysts, since no nitrates were used in the preparation, during the calcination, no NO or NO₂ will be produced. While as, for reference catalysts, due to the nitrates raw materials used, large amount of NO or NO₂ will be produced during calcination, which are environmental hazardous.

For example catalysts #8b and #8c (powder), those catalysts were prepared using a dry powder mixing preparation as follows: the catalyst materials were dry mixed without the addition of water to achieve a uniform mixture. The mixture was then dried at 125° C. followed by calcination.

The reference catalysts were prepared by using nitrate raw materials. The catalysts were formed by placing a known amount of nitrates of elements A, B, C, and D in a beaker and dissolved with known amount of deionized water. Stirring was performed until the substances dissolved. The mixtures were then dried at a temperature of about 125° C. for at least 8 hours. The cakes were then transferred to a porcelain dish and placed in a heating oven for calcination at various calcination temperatures for 6 hours. Pressing is optionally included if after calcination the catalyst material is in a powder form. The solid catalysts were crushed to powder and sieved to form a product having a particle size between 35 to 60 mesh.

The different catalysts tested are listed in Table 1 and shows the weight in grams of the elements used to form the catalyst, the composition, and calcination temperature. For example catalysts, the raw material particle sizes are also provided.

TABLE 1

| | | Calcination | | | | |
|---|---|---|---|---|---|---|
| Catalyst | Compostion | Temp (° C.) | Sr(NO₃)₂ | Ce(NO₃)₃•6H₂O | Yb(NO₃)₃•5H₂O | La(NO₃)₃•6H₂O |
| Reference #1 | Sr_{1.0}Ce_{0.9}Yb_{0.1}Oₓ | 900, 1,100 | 4.23 | 7.82 | 0.9 | |
| Reference #2 | Sr_{0.1}La_{1.0}Ce_{0.7}Oₓ | 900 | 0.43 | 0.58 | | 8.66 |
| Reference #3 | Sr_{1.0}Ce_{0.9}La_{0.5}Yb_{0.1}Oₓ | 900 | 4.02 | 7.42 | 0.85 | 4.33 |
| Example #1 (Nano) | Sr_{1.0}Ce_{0.9}Yb_{0.1}Oₓ | 1,100 | | | | |
| Example #2 (Nano) | Sr_{0.1}La_{1.0}Ce_{0.06}Oₓ | 900 | | | | |
| Example #3 (Nano) | Sr_{0.04}La_{1.0}Ce_{0.05}Oₓ | 900 | | | | |
| Example #4 (Nano) | Sr_{1.0}Ce_{0.9}La_{0.5}Yb_{0.1}Oₓ | 900 | | | | |
| Example #5 (Nano) | Sr_{1.0}La_{0.9}Nd_{0.7}Y_{0.1}Oₓ | 900 | | | | |
| Example #6 (Nano) | Sr_{1.0}La_{1.0}Yb_{0.1}Ta_{0.1}Oₓ | 800 | | | | |
| Example #7 (Nano) | Sr_{1.0}La_{1.0}Yb_{0.1}Ta_{0.1}Oₓ | 800 | | | | |
| Example #8 (Nano) | Sr_{1.0}La_{1.0}Nd_{0.7}Yb_{0.3}Oₓ | 900 | | | | |
| Example #8b (Powder) | Sr_{1.0}La_{1.0}Nd_{0.7}Yb_{0.3}Oₓ | 800 | | | | |
| Example #8c (Powder) | Sr_{1.0}La_{1.0}Nd_{0.7}Yb_{0.3}Oₓ | 650 | | | | |

| | SrCO₃ | CeO₂ | Yb₂O₃ | La₂O₃ | Nd₂O₃ | Ta₂O₅ | Nb₂O₅ | Y₂O₃ |
|---|---|---|---|---|---|---|---|---|
| Catalyst | (<800 nm) | (<25 nm) | (<80 nm) | (<100 nm) | (<100 nm) | (<100 nm) | (<100 nm) | (<100 nm) |
| Reference #1 | | | | | | | | |
| Reference #2 | | | | | | | | |
| Reference #3 | | | | | | | | |
| Example #1 (Nano) | 2.71 | 27.86 | 7.39 | | | | | |
| Example #2 (Nano) | 0.55 | 3.5 | | 6.05 | | | | |
| Example #3 (Nano) | 0.24 | 3.45 | | 6.05 | | | | |
| Example #4 (Nano) | 4.99 | 25.88 | 0.66 | 2.72 | | | | |
| Example #5 (Nano) | 5.91 | | | 11.73 | 9.42 | | | 1.58 |
| Example #6 (Nano) | 2.96 | | 0.4 | 2.93 | | 0.37 | | |
| Example #7 (Nano) | 4.43 | | 0.59 | 4.4 | | | 0.28 | |
| Example #8 (Nano) | 2.96 | | 1.19 | 2.94 | 2.36 | | | |
| Example #8b (Powder) | 5.91 | | 2.37 | 5.87 | 4.72 | | | |
| Example #8c (Powder) | 5.91 | | 2.37 | 5.87 | 4.72 | | | |

Catalysts were performance tested under two different testing conditions. For the first testing condition: 20 milligrams (mg) of the catalysts were then loaded into a 2.3 millimeter (mm) inner diameter quartz tube reactor. A feed stream of a mixture of methane and oxygen at a methane to oxygen ratio of 7.4:1 was flowed over the catalyst at a flow rate of 40 standard cubic centimeters per minute (sccm). Catalyst performances were obtained by varying the reactor temperatures. The oxygen conversion and C₂⁺ selectivity were measured using an Agilent 7890 gas chromatograph with a thermal conductivity detector and a flame ionization detector.

For the second testing condition: 30 milligrams (mg) of the catalysts were then loaded into a 4 millimeter (mm) inner diameter quartz tube reactor. A feed stream of a mixture of methane and oxygen at a methane to oxygen ratio of 4:1 was flowed over the catalyst at a flow rate of 160 sccm. Catalyst performances were obtained by varying the reactor temperatures. The oxygen conversion and C₂⁺ selectivity were measured using an Agilent 7890 gas chromatograph with a thermal conductivity detector and a flame ionization detector.

The performances of reference catalyst #1 calcined at 650° C., 900° C., and 1,100° C. under the first testing condition are shown in Table 2. It can be seen that 900° C. calcined sample shows the best activity. The performance of Example #1 catalyst calcined at 1,100° C. under the first testing conditions is also shown in Table 2. Compared to the reference catalyst #1 calcined at the same temperature, higher activities were obtained with Example #1 catalyst with higher oxygen conversions being obtained at the same testing temperature. The best selectivity obtained with the reference catalyst #1 nitrate preparation was 75.9%, and the best selectivity obtained with example catalyst #1 from nano oxides was 77.7%. This again demonstrates that better selectivity is obtained with the example #1 catalyst made from nano materials.

TABLE 2

| | | | O₂ | |
|---|---|---|---|---|
| | Calcination | Reactor | Conversion | C₂⁺ Best |
| Composition | Temp (° C.) | Temp (° C.) | (%) | Selectivity (%) |
| Reference | 900 | 700 | 26.5 | 75.4 |
| Catalyst #1 | | 750 | 93.1 | |
| | 1,100 | 700 | 13.4 | 75.9 |
| | | 750 | 63.8 | |
| Example | 1,100 | 700 | 61.2 | 77.7 |
| Catalyst #1 | | 750 | 91.1 | |

Comparison of the catalyst surface area in units of m²/g of the two preparation methods under different calcination temperatures are displayed in Table 3. It can be seen that higher surface areas are obtained with the catalysts made from nano materials with the activity gain obtained with nano materials being much higher than the surface area difference between these two methods, which indicates that there are intrinsic activity improvements achieved when nano materials are used for preparation. One possible reason could be the better interactions between different phases which are responsible for the OCM reaction with preparation with the nano materials. The OCM reaction is a multi-step reaction and requires cooperation between difference phases/sites. Therefore, a small particle size could create a better interaction between these phases/sites that can result in a better activity being obtained. It can be predicted that these improved interactions between different phases is also important for selectivity. It is theorized that better interaction between the different phases may reduce the isolated islands of single phases in some catalysts. Some of these islands may contribute to the COx formation. Therefore, reduction of such islands may lower the COx formation and enhance selectivity.

TABLE 3

| | Calcination Temp (° C.) | | |
|---|---|---|---|
| | 650 | 900 | 1,100 |
| Surface area of Example | 18.8 | 5.3 | 2.0 |
| Catalyst #1 m2/g | | | |
| Surface area of Reference | 3.9 | 2.9 | 1.6 |
| Catalyst #1 m2/g | | | |

The performance of reference catalyst #2 is shown in Table 4. This is another reference catalyst made from nitrate materials, but with a different composition compared to reference catalyst #1. Comparing the data shown in Tables 2 and 4, with reference catalyst #1 calcined at 900° C., the oxygen conversion is 26.5% at 700° C.; but with reference catalyst #2 the oxygen conversion reached 43.0% at 600° C. indicating that reference catalyst #2 reached a higher oxygen conversion at 100° C. lower temperature; therefore showing that a significant activity increase is achieved with the catalyst having the composition of reference catalyst #2. The best selectivities obtained with reference and example catalysts #2 were very comparable, about 76%. Example #2 catalyst made with nano materials, achieved the same oxygen conversion as reference catalyst #2, but at a temperature of 550° C., which is 100° C. lower than reference catalyst #2. Therefore, with the formulation of example catalyst #2 made with nano materials, the reaction temperature can be lowered by 200° C., compared to the formulation of reference catalyst #1, which is a significant improvement. Again, the selectivity is comparable for the catalysts in Tables 2 and 4.

TABLE 4

| | | | O₂ | |
|---|---|---|---|---|
| | Calcination | Reactor | Conversion | C₂⁺ Best |
| Composition | Temp (° C.) | Temp (° C.) | (%) | Selectivity (%) |
| Reference | 900 | 600 | 43.0 | 76.6 |
| Catalyst #2 | | 650 | 69.3 | |
| Example | 900 | 550 | 68.7 | 75.4 |
| Catalyst #2 | | | | |

The performance of example #3 catalyst is listed in Table 5. As can be seen, the activity obtained with example #3 catalyst is higher than example #2 catalyst; therefore, the catalyst activity can be increased further by optimizing the catalyst composition. The selectivity obtained with example #3 catalyst is comparable to that obtained by example #2 catalyst.

TABLE 5

| | | | O₂ | |
|---|---|---|---|---|
| | Calcination | Reactor | Conversion | C₂⁺ Best |
| Composition | Temp (° C.) | Temp (° C.) | (%) | Selectivity (%) |
| Example | 900 | 550 | 76.2 | 76.2 |
| Catalyst #3 | | | | |

The performance of reference catalyst #3 and example catalyst #4 are shown in Table 6. Reference catalyst #3 is another reference catalyst made from nitrate materials, but with a different composition. Example catalyst #4 was made with nano materials. Comparing the data shown in Tables 2 and 6, for reference catalyst #1 calcined at 900° C., the oxygen conversion was 26.5% at 700° C., but with reference catalyst #3, the oxygen conversion reached 44.8% at 650° C. indicating that the formulation of reference catalyst #3 had a much higher oxygen conversion at a temperature that was 50° C. lower than reference catalyst #1. Therefore, a significant activity increase can be achieved with the catalyst having the composition of reference catalyst #3. As can also be seen, example catalyst #4 made with nano materials exhibited a higher oxygen conversion at 550° C. compared to reference catalyst #3. Thus, the reaction temperature can be reduced by another 100° C. from the reference catalyst #3 to get a better oxygen conversion. Moreover, the formulation of the example #4 catalyst made with nano materials can be used to lower the reaction temperature by 150° C. compared to reference catalyst #1, indicating a significant activity improvement. Again, the selectivity obtained for example catalyst #4 is comparable to reference catalyst #3.

TABLE 6

| | | | O₂ | |
|---|---|---|---|---|
| | Calcination | Reactor | Conversion | C₂⁺ Best |
| Composition | Temp (° C.) | Temp (° C.) | (%) | Selectivity (%) |
| Reference | 900 | 650 | 44.8 | 78.2 |
| Catalyst #3 | | | | |
| Example | 900 | 550 | 56.0 | 77.3 |
| Catalyst #4 | | | | |

The performance of the example catalyst #5 is shown in Table 7. Compared to the reference catalyst #1 calcined at 900° C., it can be seen that higher activity and selectivity are obtained with example catalyst #5.

TABLE 7

| | | | O₂ | |
|---|---|---|---|---|
| | Calcination | Reactor | Conversion | C₂⁺ Best |
| Composition | Temp (° C.) | Temp (° C.) | (%) | Selectivity (%) |
| Example | 900 | 700 | 87.4 | 79.1 |
| Catalyst #5 | | 750 | 100 | |

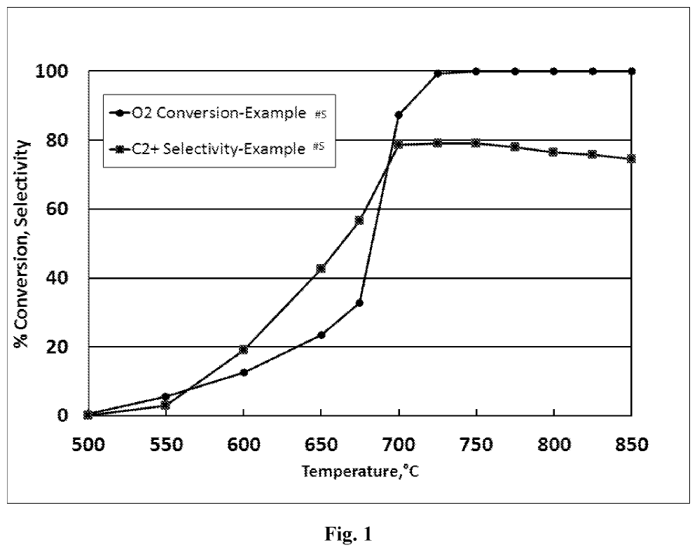

FIG. 1 is a graph of the oxygen conversion and C₂+ selectivity versus reactor temperature for catalyst example #5. It can be seen that with this catalyst, the O₂ conversion reaches higher than 90% at 725° C. As can also be seen in FIG. 1, the C₂+ selectivity is related to the reaction temperature and the O₂ conversion. The best selectivity obtained with catalyst example #5 is 79.1%. Therefore, this catalyst obtained excellent O₂ conversion and C₂+ selectivity at a lower reactor temperature.

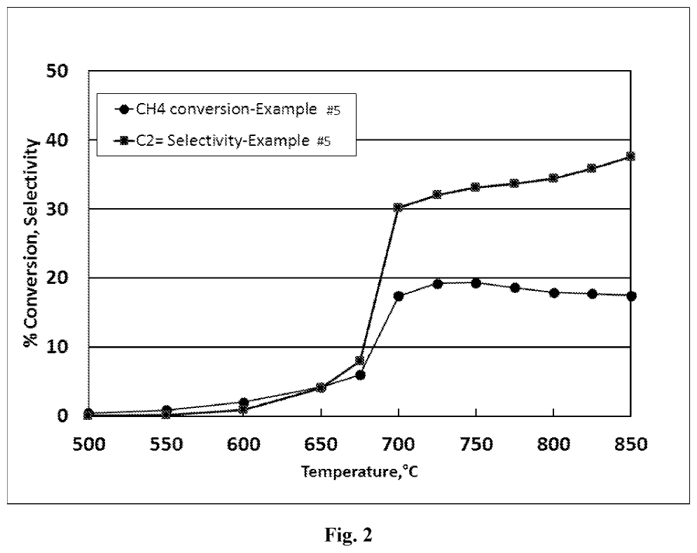

FIG. 2 is a graph of the methane conversion and ethylene selectivity versus reactor temperature for catalyst example #5. Ethylene is the most important and desirable product in an OCM product stream. The best ethylene selectivity obtained with this catalyst is 37.6%.

Table 8 compared the reactor temperature, C₂+ selectivity, and ethylene selectivity for various catalysts. Example catalysts #5-#8 obtained good results with nano raw materials, in which B, C, D are nano oxides and A is nano carbonate. Catalyst example #7 obtained the highest C₂+ selectivity, but at a higher reactor temperature and lower ethylene selectivity.

Comparing example #8 and #8b, where #8 was prepared in a slurry form and #8b was prepared in a dry powder mixing form; a higher catalyst activity can be obtained with no change in the selectivity at a lower reactor temperature when using the dry powder mixing form. This indicates that the catalyst activity can be improved further by using the dry powder mixing form for some of the catalysts which are prepared by using the nano materials. Comparing example #8b and example #8c, where the calcination temperature for #8c was lower than #8b it can be seen that for the dry powder mixing method, a higher calcination temperature is needed to achieve a better performance.

TABLE 8

| Catalyst | reach >90% O₂ | Selectivity | Selectivity |
|---|---|---|---|
| Example # | Conver. | (%) | (%) |
| 5 | 725 | 79.1 | 37.6 |
| 6 | 750 | 76.8 | 37.8 |
| 7 | 775 | 80.1 | 39.3 |
| 8 | 725 | 78.7 | 37.1 |
| 8b | 675 | 78.6 | 36 |
| 8c | 700 | 66.2 | 29.4 |

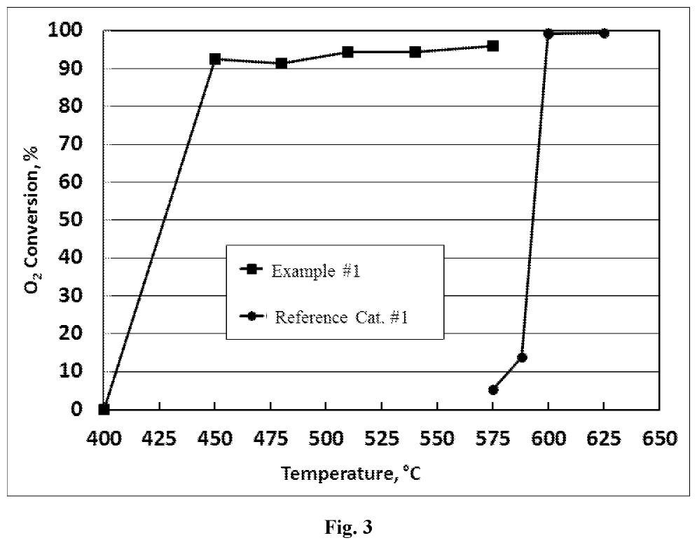

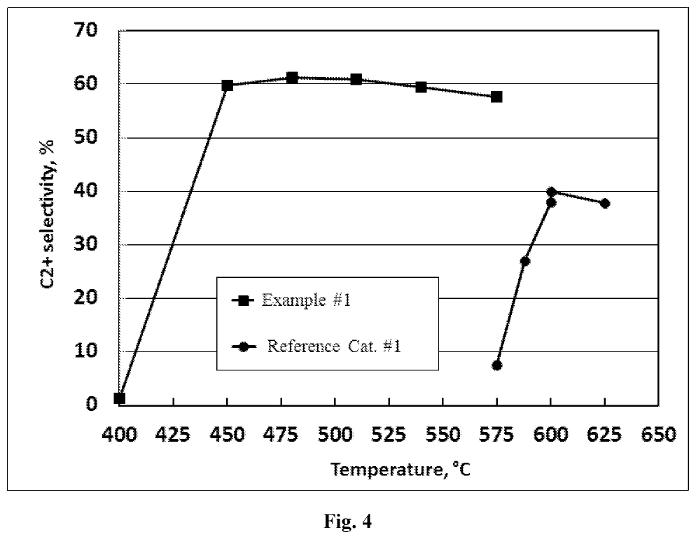

Example #1 which was calcined at 650° C. and reference catalyst #1 which was calcined at 900° C. are tested with the second testing conditions. The results obtained are shown in FIGS. 3 and 4. It can be seen that example #1 reached 90% or higher oxygen conversion at 450° C., while the reference catalyst #1 reached 90% or higher oxygen conversion at 600° C., indicating a much higher activity can be obtained with example #1 which is made from nano oxide materials. It clearly demonstrated that example #1 catalyst can be used to catalyze the OCM reaction at a very low feed temperature, so that it is suitable for adiabatic operation with low feed temperature.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is, therefore, evident that the particular illustrative embodiments disclosed above may be altered or modified and all such variations are considered within the scope and spirit of the present invention.

As used herein, the words “comprise,” “have,” “include,” and all grammatical variations thereof are each intended to have an open, non-limiting meaning that does not exclude additional elements or steps. While compositions and methods are described in terms of “comprising,” “containing,” or “including” various components or steps, the compositions and methods also can “consist essentially of” or “consist of” the various components and steps. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent(s) or other documents that may be incorporated herein by reference, the definitions that are consistent with this specification should be adopted.

What is claimed is:

1. A catalytic material for oxidative coupling of methane comprising:
a catalyst with the formula AₐB_{b}C_{c}Oₓ, wherein:
A is selected from alkaline earth metals;
B and C are selected from rare earth metals, and wherein
B and C are different rare earth metals; and
the oxide of at least A, B, and C has basic, redox, or both basic and redox properties, and wherein the elements A, B, and C are selected to create a synergistic effect whereby the catalytic material provides an oxygen conversion of greater than or equal to 50% and a C₂⁺ selectivity of greater than or equal to 70%, and wherein the catalyst provides the oxygen conversion and selectivity at a temperature of 797° F. (425° C.) or greater.

2. The catalytic material according to claim 1, wherein the catalyst is thermally stable at a temperature of 797° F. (425° C.) or greater.

3. The catalytic material according to claim 1, wherein the catalyst is thermally stable at a temperature in the range of about 797° F. (425° C.) to about 2,372° F. (1,300° C.).

4. The catalytic material according to claim 1, wherein the catalyst provides the oxygen conversion and selectivity at a temperature in the range of about 797° F. (425° C.) to about 2,012° F. (1,100° C.).

5. The catalytic material according to claim 1, wherein: a=1.0; b and c are each in the range from about 0.01 to about 10; and x is a number selected to balance the oxidation states of A, B, and C.

6. The catalytic material according to claim 1, wherein A, B, and C and the ratios of A, B, and C are selected to provide an oxygen conversion of greater than or equal to 50% and a C₂⁺ selectivity of greater than or equal to 70% to the catalytic material.

7. The catalytic material according to claim 1, whereby the catalytic material provides an oxygen conversion of greater than or equal to 90% and a C₂⁺ selectivity of greater than or equal to 75%.

8. The catalytic material according to claim 1, wherein the raw materials used for the catalyst preparation of the catalytic material are nano materials.

9. The catalytic material according to claim 1, wherein:
A is selected from the group consisting of magnesium, calcium, strontium, and barium; and
B and C are selected from the group consisting of lanthanum, scandium, cerium, praseodymium, neodymium, promethium, samarium, europium, gadolinium, yttrium, terbium, dysprosium, holmium, erbium, thulium, ytterbium, and lutetium.

10. The catalytic material according to claim 1, wherein at least one of B and C has redox properties.

11. The catalytic material according to claim 1, wherein the catalytic material is used in an adiabatic rector.

12. The catalytic material according to claim 1, further comprising D_{d} wherein _{d} is in the range from about 0 to about 10.

13. The catalytic material according to claim 12, wherein D is selected from the group consisting of manganese, tungsten, bismuth, antimony, niobium, tantalum, iron, copper, or a rare earth metal, wherein if D is selected from a rare earth metal, then D is a different rare earth metal from B and C

14. A system for oxidative coupling of methane comprising:
a source of methane;
a source of oxygen;
a catalytic material, wherein the catalytic material comprises
a catalyst with the formula AₐB_{b}C_{c}Oₓ, and wherein:
A is selected from alkaline earth metals;
B and C are selected from rare earth metals, and wherein
B and C are different rare earth metals; and
the oxide of at least A, B, and C has basic, redox, or both basic and redox properties, and wherein the elements A, B, and C are selected to create a synergistic effect whereby the catalytic material provides an oxygen conversion of greater than or equal to 50% and a C₂⁺ selectivity of greater than or equal to 70%, and wherein the catalyst provides the oxygen conversion and selectivity at a temperature of 797° F. (425° C.) or greater, and
wherein the catalytic material produces ethane, ethylene, or combinations thereof; and
a device for collecting the ethane, ethylene, or combinations thereof.

15. The system according to claim 14, wherein the ratio of methane to oxygen is selected to provide a percent yield of the ethane, ethylene, or combinations thereof that is greater than or equal to 10%.

16. The system according to claim 14, wherein the ratio of methane to oxygen is selected to provide a percent yield of the ethane, ethylene, or combinations thereof that is greater than or equal to 15%.

17. A method for the oxidative coupling of methane comprising:
providing a source of methane;
providing a source of oxygen;
contacting the source of methane and the source of oxygen with
a catalytic material, wherein the catalytic material comprises
a catalyst with the formula AₐB_{b}C_{c}Oₓ, and wherein:
A is selected from alkaline earth metals;
B and C are selected from rare earth metals, and wherein
B and C are different rare earth metals; and
the oxide of at least A, B, and C has basic, redox, or both basic and redox properties, and wherein the elements A, B, and C are selected to create a synergistic effect whereby the catalytic material provides an oxygen conversion of greater than or equal to 50% and a C₂⁺ selectivity of greater than or equal to 70%, and wherein the catalyst provides the oxygen conversion and selectivity at a temperature of 797° F. (425° C.) or greater, and
wherein the catalytic material produces ethane, ethylene, or combinations thereof after contact with the source of methane and the source of oxygen; and
collecting the ethane, ethylene, or combinations thereof.

18. The method according to claim 17, wherein the ratio of methane to oxygen is selected to provide a percent yield of the ethane, ethylene, or combinations thereof that is greater than or equal to 10%.

19. The method according to claim 17, wherein the ratio of methane to oxygen is selected to provide a percent yield of the ethane, ethylene, or combinations thereof that is greater than or equal to 15%.
